(19) United States
(12) Patent Application Publication
(10) Pub. No.: US 2004/0054227 A1
(43) Pub. Date: Mar. 18, 2004

(54) Use of compositions contanining urea derivatives of cyanoacetic acid and urea as stabilisers for chlorine-containing thermoplastic plastics

(76) Inventors: Peter Daute, Beverstedt (DE); Joerg-Dieter Klamann, Bremerhaven (DE); Ralf Picard, Bremerhaven (DE); Dieter Marks, Loxstedt (DE); Peter Wedl, Bremerhaven (DE)

Correspondence Address: COGNIS CORPORATION, PATENT DEPARTMENT, 300 BROOKSIDE AVENUE, AMBLER, PA, 19002, US

(21) Appl. No.: 10/312,286

(22) PCT Filed: Jun. 12, 2001
(86) PCT No.: PCT/EP01/06605

(30) Foreign Application Priority Data
Jun. 21, 2000 (DE) 100304648

(51) Int. Cl. C07C 211/00

(52) U.S. Cl. 564/1

(57) ABSTRACT

The invention relates to the use of compositions containing urea derivatives of cyanoacetic acid and urea as stabilisers for chlorine-containing thermoplastic plastics. Said derivatives are compounds of general formula (A), NC—CH₂—CO—N(R¹)—CO—NH—R₂, where R¹ and R²=independently, a branched or unbranched, linear or cyclic, alkyl group with 1 to 18 C atoms, an aryl group with 6 to 18 C atoms, optionally substituted by one or more alkyl groups with 1 to 6 C atoms.

### FIELD OF THE INVENTION

This invention relates to the use of compositions containing a combination of special urea derivatives of cyanoacetic acid and urea as stabilizers for chlorine-containing thermoplastics.

### PRIOR ART

It is known that halogen-containing plastics or molding compositions produced from them tend to undergo degradation or decomposition reactions on exposure to heat stress or high-energy radiation, for example ultraviolet light.

The stabilizing of PVC during processing has generally involved the use of metal-containing stabilizers based on Pb, Ba, Cd, Sn, Ca and Zn. As long ago as 1940, urea derivatives, such as diphenylthiourea for example, were proposed for stabilizing PVC (cf. Gächter/Müller, “Kunststoff-Additive”, Carl Hanser Verlag 1989, p. 312). These compounds are generally used in combination with metal-containing stabilizers because the long-term stability they provide is mostly inadequate.

EP-A 768 336 describes stabilizer combinations for chlorine-containing polymers, more especially polyvinyl chloride (PVC), which contain special heterocyclic compounds, namely substituted uracil derivatives. These compounds have high melting points (above 300° C.) and can only be used in fine-particle form in the processing of PVC.

EP-A-962 491 (Witco) describes the use of cyanoacetyl ureas for stabilizing halogen-containing polymers.

### DESCRIPTION OF THE INVENTION

The problem addressed by the present invention was to provide stabilizers for halogen-containing plastics. These stabilizers would be distinguished by an improved action profile in relation to the prior art. More particularly, correspondingly stabilized halogen-containing plastics would have improved initial color values under heat stress. In addition, the stabilizers would have the following properties: high compatibility with calcium and/or zinc compounds; ready incorporation in halogen-containing plastics without adversely affecting their Theological properties; good long-term stability of correspondingly stabilized halogen-containing plastics, especially PVC (polyvinyl chloride).

It has now been found that, besides a favorable initial color, compositions containing on the one hand compounds a) formed by reaction of substituted ureas with cyanoacetic acid and, on the other hand, urea b) also provide adequate long-term stability in the stabilization of chlorine-containing organic plastics. By comparison with the individual components a) and b), compositions containing both a) and b) are distinguished by a synergistic improvement in their stabilizing properties for halogen-containing polymers.

The present invention relates to the use of compositions containing a) urea derivatives of cyanoacetic acid and b) urea as stabilizers for chlorine-containing thermoplastics. The urea derivatives are substances corresponding to general formula (A):

NC—CH₂—CO—N(R¹)—CO—NH—R² (A)

in which R¹ and R² independently of one another represent an unbranched or branched, linear or cyclic alkyl group containing 1 to 18 carbon atoms or an aryl group containing 6 to 18 carbon atoms which may optionally be substituted by one or more C₁₋₆ alkyl groups.

The compositions containing a) and b) on the one hand are distinguished by the fact that they are capable of excellently stabilizing halogen-containing organic plastics, especially PVC, more particularly against thermal degradation.

On the other hand, the compositions containing a) and b) facilitate application in the plastic to be processed by virtue of the fact that they are present in liquid form at temperatures of around 180° C. at which PVC is normally processed.

According to the invention, substances (A) in which the substituents R¹ and R² independently of one another are methyl, ethyl and phenyl are preferably used. In a particularly preferred embodiment, both R¹ and R² represent methyl. This special compound is known from the literature as dimethyl(cyanoacetyl)urea.

The compositions containing a) and b) are added to halogen-containing plastics in quantities of preferably 0.01 to 10.0 parts by weight per 100 parts by weight of halogen-containing plastic.

Components a) and b) are present in the compositions in a ratio by weight of a) to b) of 2:1 to 1:5, a ratio in the 2:1 to 1:3 range being preferred.

The compounds (A) may be produced by synthesis methods known per se. These are known to the expert. More particularly, the compounds (A) are produced by reaction of the corresponding urea derivatives with cyanoacetic acid.

Component (B) consists of urea (melting point 133° C.) which is available in large quantities as an industrial raw material and which is produced by generally known methods,

In one embodiment, the compositions containing a) and b) to be used in accordance with the invention are used in combination with one or more additives selected from the group consisting of

(C1) perchlorate compounds,

(C2) glycidyl compounds,

(C3) β-diketones and β-ketoesters,

(C4) dihydropyridines and polydihydropyridines,

(C5) polyols and disaccharide alcohols,

(C6) sterically hindered amines (tetraalkyl piperidine compounds),

(C7) alkali metal alumosilicates (zeolites),

(C8) hydrotalcites and modified hydrotalcites,

(C9) alkali metal alumocarbonates (dawsonite),

(C10) alkali metal and alkaline earth metal hydroxides, hydrogen carbonates and carbonates,

(C11) antioxidants,

(C12) release agents and/or lubricants,

(C13) plasticizers,

(C14) pigments,

(C15) fillers,

(C16) phosphites,

(C17) thiophosphites and thiophosphates,

(C18) mercaptocarboxylic acid esters,

(C19) epoxidized fatty acid esters,

(C20) UV absorbers and light stabilizers,

(C21) blowing agents,

(C22) organozinc compounds,

(C23) organotin compounds.

The compounds of classes (C1) to (C23) are well-known to the expert as additives for halogen-containing plastics, especially PVC. For representative examples of substances belonging to these classes, reference is specifically made to EP-A-768 336.

In connection with the use of the term “additive”, it is pointed out that the expert on the processing of plastics classifies the additives both from the structural and from the functional perspective.

Under the functional classification, typical additives for plastics are antistatic agents, antifogging agents, antioxidants, UV stabilizers, coupling agents, calendering aids, mold release agents, slip agents, parting agents, lubricants, plasticizers, fragrances, flame retardants, fillers, pigments, blowing agents, agents for increasing thermal stability (heat stabilizers).

The above-mentioned additive classes (C1) to (C23) largely follow the structural classification, i.e. the classification based on chemical structure. In the case of classes (C11) to (C15) and (C20) and (C21), however, the functional definition was preferred.

In addition, it is pointed out that compounds belonging to a certain class, i.e. compounds which may be assigned to the same class from the structural perspective, often perform not just one, but two or more functions in practice. For example, calcium or zinc soaps can act as slip agents and/or parting agents, but may also be used as heat stabilizers, for example in the processing of polyvinyl chloride (PVC).

Examples of suitable compounds from the group of substances belonging to group (C1) can be found on page 3, lines 32 to 43 of the above-cited EP-A-768 336.

Examples of suitable compounds from the group of substances belonging to group (C2) can be found on page 3, lines 47 to 57 and on page 4, line 57 to page 6, line 32 of the above-cited EP-A-768 336.

Examples of suitable compounds from the group of substances belonging to group (C3) can be found on page 6, lines 7 to 24 of the above-cited EP-A-768 336.

Examples of suitable compounds from the group of substances belonging to group (C5) can be found on page 7, lines 14 to 20 of the above-cited EP-A-768 336.

Examples of suitable compounds from the group of substances belonging to group (C7) can be found on page 25, line 54 to page 27, line 15 of the above-cited EP-A-768 336.

Examples of suitable compounds from the group of substances belonging to group (C8) can be found on page 25, lines 25 to 50 of the above-cited EP-A-768 336.

Examples of suitable compounds from the group of substances belonging to group (C10) can be found on page 28, line 56 to page 29, line 6 of the above-cited EP-A-768 336.

With regard to the substances belonging to group (C12), it is expressly pointed out that both slip agents and parting agents and mixtures of slip and parting agents may be used. In the standard language of the expert, parting agents are products which reduce the frictional resistances mainly between the polymer melt and the steel surface of the machine used for moulding/processing. The effect of the reduced frictional resistance is that the melt pressure is reduced. By contrast, slip agents mainly act in the polymer melt and reduce the internal friction forces so that, even with high filler contents, the melt retains good plastic flow which is important for filling the mould.

In one embodiment of the invention, the slip or parting agents used are calcium salts and/or magnesium salts and/or aluminium salts solid or liquid at 20° C. which are selected from

calcium salts of saturated or unsaturated, linear or branched monocarboxylic acids containing 6 to 36 carbon atoms,

calcium salts of unsubstituted or C₁₋₄-alkyl-substituted benzoic acid,

zinc salts of saturated or unsaturated, linear or branched monocarboxylic acids containing 6 to 36 carbon atoms,

magnesium salts of saturated or unsaturated, linear or branched monocarboxylic acids containing 6 to 36 carbon atoms,

magnesium salts of saturated or unsaturated dicarboxylic acids containing 6 to 10 carbon atoms,

aluminium salts of saturated or unsaturated, linear or branched monocarboxylic acids containing 6 to 36 carbon atoms.

The above-mentioned calcium, magnesium, zinc and aluminium salts may be used both individually and in admixture with one another.

Other slip or parting agents which may be used individually or in combination with one another as component (B12) are the substances known from the relevant prior art. Compounds of the following types are preferred: hydrocarbon waxes melting at temperatures of 70 to 130° C., oxidized polyethylene waxes, free fatty acids containing 8 to 22 carbon atoms and branched-chain isomers thereof, for example stearic acid or even hydroxystearic acid, α-olefins, wax esters, i.e. esters of relatively long-chain monocarboxylic acids and monoalcohols, primary and secondary, saturated and unsaturated higher alcohols preferably containing 16 to 44 carbon atoms in the molecule, ethylenediamine distearate, montanic acid esters of diols, for example ethanediol, butane-1,3-diol and glycerol, mixtures of such montanic acid esters with nonesterified montanic acids, partial esters of fatty acids containing 8 to 22 carbon atoms and polyols containing 2 to 6 carbon atoms and 2 to 6 hydroxyl groups which contain on average at least one free polyol hydroxyl group per molecule. Also suitable are the mixed esters of aliphatic, cycloaliphatic or aromatic dicarboxylic acids containing 2 to 22 carbon atoms in the molecule, aliphatic polyols containing 2 to 6 hydroxyl groups in the molecule and aliphatic monocarboxylic acids containing 12 to 30 carbon atoms in the molecule described in DE-C-19 07 768 with hydroxyl or acid values of 0 to 6. Examples include mixed esters of maleic acid/pentaerythritol/behenic acid, mixed esters of adipic acid/pentaerythritol/oleic acid and mixed esters of adipic acid/pentaerythritol/stearic acid. According to the invention, slip or parting agents of this type may be used individually and in combination with one another and with the above-mentioned calcium, magnesium, zinc or aluminium salts.

Examples of suitable compounds from the group of substances belonging to group (C13) can be found on page 29, line 22 to page 30, line 26 of the above-cited EP-A-768 336.

Among the pigments (C14), titanium dioxide is preferred.

Among the fillers (C15), calcium carbonate (chalk), talcum, kaolin and the like are preferred. Chalk is most particularly preferred.

Examples of suitable compounds from the group of substances belonging to group (C22) can be found on page 28, lines 13 to 45 of the above-cited EP-A-768 336.

The present invention also relates to stabilizer compositions for chlorine-containing thermoplastics containing substances corresponding to general formula (A):

NC—CH₂—CO—N(R¹)—CO—NH—R² (A)

in which R¹ and R² independently of one another represent an unbranched or branched, linear or cyclic alkyl group containing 1 to 18 carbon atoms or an aryl group containing 6 to 18 carbon atoms which may optionally be substituted by one or more C₁₋₆ alkyl groups, and urea.

In another embodiment, the stabilizer compositions according to the invention contain one or more compounds from the above-mentioned additive classes (C1) to (C23).

The present invention also relates to a process for stabilizing halogen-containing organic plastics in which compositions containing substances corresponding to general formula (A):

NC—CH₂—CO—N(R¹)—CO—NH—R² (A)

in which R¹ and R² independently of one another represent an unbranched or branched, linear or cyclic alkyl group containing 1 to 18 carbon atoms or an aryl group containing 6 to 18 carbon atoms which may optionally be substituted by one or more C₁₋₆ alkyl groups, and urea are incorporated in the chlorine-containing plastics.

### EXAMPLES

### Example 1

### Preparation of dimethyl(cyanoacetyl)urea by Condensation of N,N-dimethylurea With Cyanoacetic Acid

Equimolar quantities of N,N-dimethylurea (56.8 g) and cyanoacetic acid (54.8 g) were heated to 80° C. and 65.8 g of acetic anhydride were then introduced over a period of 20 minutes. The temperature was kept at 90° C. for another 90 minutes and the acetic acid was distilled off in vacuo (ca. 20 mbar). A slowly solidifying yellow oil was obtained. The melting point of the pure compound was found to be 77.5-78.5° C.

### APPLICATION EXAMPLES

PVC Stabilizer Mixtures (Various Urea Derivatives):

The composition of the test formulations B2 to B7 is given in the following (in parts by weight). Test formulation B4 corresponds to the invention, the other Examples are intended for comparison.

| | B2 | B3 | B4 | B5 | B6 | B7 |
|---|---|---|---|---|---|---|
| PVC (Evipol | 100 | 100 | 100 | 100 | 100 | 100 |
| SH 6030, | | | | | | |
| manufacturer: | | | | | | |
| EVC) | | | | | | |
| Chalk | 3.0 | 3.0 | 3.0 | 3.0 | 3.0 | 3.0 |
| (Omyalite | | | | | | |
| 95 T) | | | | | | |
| Zeolite A | 0.84 | 0.84 | 0.84 | 0.84 | 0.84 | 0.84 |
| Magnesium | 0.4 | 0.4 | 0.4 | 0.4 | 0.4 | 0.4 |
| stearate | | | | | | |
| Sodium | 0.05 | 0.05 | 0.05 | 0.05 | 0.05 | 0.05 |
| perchlorate | | | | | | |
| Compound of | — | 0.2 | 0.2 | 0.2 | 0.2 | 0.2 |
| Example 1 | | | | | | |
| Urea | — | — | 0.2 | — | — | — |
| Diphenyl- | — | — | | 0.2 | — | — |
| thiourea | | | | | | |
| Diphenylurea | — | — | — | — | 0.2 | — |
| N,N′- | — | — | — | — | — | 0.2 |
| dimethylurea | | | | | | |

Strips were produced from the formulations and tested for static thermal stability at 180° C. The strips were produced by homogenizing and plasticizing the PVC powder mixture and the additives mentioned for 5 minutes at 170° C. on a laboratory roll mill. Test specimens measuring 17×17 mm were cut out from the ca. 0.5 mm thick strips thus produced.

The test specimens were placed in a heating cabinet at 180° C. on glass plates on rotating trays and removed at 15-minute intervals until all the test specimens were “burnt” (i.e. were black in color).

| | Initial | Color after | Color after | Color after | Color after |
|---|---|---|---|---|---|
| Example | color | 15 mins. | 30 mins. | 45 mins. | 60 mins. |
| B2 | Reddish | Brown | Brown | Dark | |
| | | | | brown | |
| B3 | Yellowish | Light | Brown | Dark | |
| | | brown | | brown | |
| B4 | Almost | Yellowish | Light | Light | Dark |
| | colorless | | brown | brown | brown |
| B5 | Colorless | Light | Brown | Dark | |
| | | brown | | brown | |
| B6 | Almost | Light | Brown | Dark | |
| | colorless | brown | | brown | |
| B7 | Almost | Light | Brown | Dark | |
| | colorless | brown | | brown | |

It can be seen from the Table that Example B4 according to the invention shows significantly better behavior than all the Comparison Examples.

PVC Stabilizer Mixtures (Variation of the Quantity of Urea):

The composition of test formulations B8 to B11 is shown in the following (in parts by weight).

| | B8 | B9 | B10 | B11 |
|---|---|---|---|---|
| PVC (Evipol SH 6030, EVC) | 100 | 100 | 100 | 100 |
| Chalk (Omyalite 95 T) | 3.0 | 3.0 | 3.0 | 3.0 |
| Zeolite A | 0.84 | 0.84 | 0.84 | 0.84 |
| Magnesium stearate | 0.4 | 0.4 | 0.4 | 0.4 |
| Sodium perchlorate | 0.05 | 0.05 | 0.05 | 0.05 |
| Compound of Example 1 | 0.2 | 0.2 | 0.2 | 0.2 |
| Urea | 0.1 | 0.3 | 0.4 | 0.5 |

Strips were produced from formulations B4 (see above) and B8 to B11 and were tested for static thermal stability at 180° C. The strips were produced as described above.

| | Initial | Color after | Color after | Color after | Color after |
|---|---|---|---|---|---|
| Example | color | 15 mins. | 30 mins. | 45 mins. | 60 mins. |
| B8 | Yellowish | Light | Light | Brown | Dark |
| | | brown | brown | | brown |
| B4 | Almost | Yellowish | Light | Light | Dark |
| | colorless | | brown | brown | brown |
| B9 | Almost | Yellowish | Light | Light | Brown |
| | colorless | | brown | brown | |
| B10 | Almost | Yellowish | Light | Light | Brown |
| | colorless | | brown | brown | |
| B11 | Almost | Yellowish | Light | Brown | Brown |
| | colorless | | brown | | |

Summary of the Tests of Examples B2 to B11:

It was found that the test formulations containing the compound of Example 1 according to the invention and urea provided PVC with distinctly better heat stabilization (initial color, color holding) than did the other formulations (Comparison Examples).

Determination of Yellow Values:

In addition, the L*,a*,b*-method (cf. DIN 6174) known to the expert was applied to the test strips used for further characterization. The b* value indicates the position on the blue/yellow axis. Normally, the b* value is also known as the yellow value. A commercially available instrument (Dr. Lange “Micro Color”) was used for the measurements. To this end, the test strips containing the test substances were heated at 180° C. in a thermo-oven, the test strip being briefly removed from the oven at 15-minute intervals. The test results are set out in Table 1.

TABLE 1
b* values (yellow values to DIN 6174)

| Example | After 0 mins. | After 15 mins. |
|---|---|---|
| B3 | 29 | 57 |
| B4 | 18 | 43 |
| B5 | 11 | 52 |
| B6 | 23 | 49 |
| B7 | 16 | 51 |
| B9 | 14 | 42 |
| B10 | 11 | 39 |
| V11 | 12 | 41 |

Determination of the Congo Red Value:

In addition, the Congo Red method known to the expert (Euro Standard EN 60811-3-2:1995, para. 9) was applied to the test strips for further characterization.

To this end, small samples (50±5 mg) were taken from the strips and heated to 200° C. (±0.5° C.) in the corresponding glass tubes in a metal block. A strip of universal indicator paper was inserted into the upper end of the glass tube. The time taken by the color of the indicator paper to just change to red was measured in minutes.

TABLE 2
Congo Red Test (temperature: 200° C.)

| Example | HCl evolution [mins] |
|---|---|
| B2 | 10 |
| B3 | 11 |
| B4 | 14 |
| B5 | 12 |
| B6 | 12 |
| B7 | 11 |
| B9 | 15 |
| B10 | 15 |
| B11 | 15 |

It can be seen from the Table that significantly better results were obtained with Examples B4, B9, B10 and B11 according to the invention than with the Comparison Examples.

1. The use of compositions containing urea derivatives of cyanoacetic acid a) and urea b) as stabilizers for chlorine-containing thermoplastics, the compounds a) used being substances corresponding to general formula (A):
NC—CH₂—CO—N(R¹)—CO—NH—R² (A)
in which R¹ and R² independently of one another represent an unbranched or branched, linear or cyclic alkyl group containing 1 to 18 carbon atoms or an aryl group containing 6 to 18 carbon atoms which may optionally be substituted by one or more C₁₋₆ alkyl groups.

2. The use claimed in claim 1, characterized in that substance (A) is dimethyl(cyanoacetyl)urea.

3. The use claimed in claim 1 or 2, characterized in that the ratio by weight of components a) and b) in the compositions is 2:1 to 1:3.

4. Stabilizer compositions for chlorine-containing thermoplastics containing substances corresponding to general formula (A):
NC—CH₂—CO—N(R¹)—CO—NH—R² (A)
in which R¹ and R² independently of one another represent an unbranched or branched, linear or cyclic alkyl group containing 1 to 18 carbon atoms or an aryl group containing 6 to 18 carbon atoms which may optionally be substituted by one or more C₁₋₆ alkyl groups, and urea.

5. Stabilizer compositions as claimed in claim 4, characterized in that substance (A) is dimethyl(cyanoacetyl)urea.

6. A process for stabilizing halogen-containing organic plastics in which compositions containing substances corresponding to general formula (A):
NC—CH₂—CO—N(R¹)—CO—NH—R² (A)
in which R¹ and R² independently of one another represent an unbranched or branched, linear or cyclic alkyl group containing 1 to 18 carbon atoms or an aryl group containing 6 to 18 carbon atoms which may optionally be substituted by one or more C₁₋₆ alkyl groups, and urea are incorporated in the chlorine-containing plastics.

7. A process as claimed in claim 6, characterized in that substance (A) is dimethyl(cyanoacetyl)urea.
